(19) United States
(12) Patent Application Publication
(10) Pub. No.: US 2019/0125930 A1
(43) Pub. Date: May 2, 2019

(54) BIOSCAFFOLD, METHOD FOR PRODUCING THE SAME, AND USES THEREOF

(71) Applicant: NATIONAL CHENG KUNG UNIVERSITY, Tainan City (TW)

(72) Inventors: Ho-Yi TUAN-MU, New Taipei City (TW); Yi-Hao CHANG, New Taipei City (TW); Jin-Jia HU, Tainan City (TW)

(73) Assignee: NATIONAL CHENG KUNG UNIVERSITY, Tainan City (TW)

(21) Appl. No.: 15/795,236

(22) Filed: Oct. 26, 2017

(51) Int. Cl. A61L 27/36; A61L 27/38

(52) U.S. Cl. CPC A61L 27/3687; A61L 2430/40; A61L 27/38; A61L 27/3604

(57) ABSTRACT

Disclosed herein is a method of producing a decellularized bioscaffold from an organ or a vessel covered by a lining tissue. The method comprises subjecting the organ or the vessel to a digestion buffer thereby removing the lining tissue from the tissue or organ; and then treating the tissue or organ with a decellularization buffer. The present decellularized bioscaffold is useful in promoting tissue regeneration and/or remodeling.

### BACKGROUND OF THE INVENTION

### 1. Field of the Invention

The present disclosure in general relates to the field of tissue engineering. More particularly, the present disclosure relates to a decellularized bioscaffold, the manufacture and uses thereof.

### 2. Description of Related Art

Decellularization is a process that removes cellular contents from a tissue or an organ, while minimizing adverse effects on the composition, biological activity and mechanical integrity of the extracellular matrix (ECM). Biological scaffolds derived from decellularized tissues or organs have been commonly and successfully used in both animal studies and human clinical applications. Compared with man-made scaffolds, decellularized biological scaffolds possess several advantages, including high biocompatibility, low immunogenicity and intrinsic mechanical competence.

In general, the cellular contents of a tissue or an organ may be removed by physical, chemical or enzymatic methods. The physical method generally involves treatments associated with temperature (i.e., removing cells and undesirable components by freeze-thaw procedure followed by treatment of liquidized chemicals), force and pressure (i.e., use of hydrostatic pressure to the tissue or organ), as well as electrical disruption (i.e., removing cells by exposing the tissue or organ to electrical pulses, which create micropores at the cell membrane). The chemicals used to kill and remove cells include acids (e.g., peracetic acid, cholic acid or acetic acid), alkalis (e.g., NaOH or ammonium salt), ionic surfactants (e.g., sodium dodecyl sulfate (SDS) or cetyltrimethylammonium bromide (CTAB)), non-ionic surfactants (e.g., Triton X-100 or Triton X-114), and zwitterionic surfactants (e.g., 3-[(3-cholamidopropyl)dimethylammonio]-1-propanesulfonate (CHAPS) or 3-[(3-Cholamidopropyl)dimethylammonio]-2-hydroxy-1-propansulfonate (CHAPSO)). The enzymes suitable for decellularization purpose include lipase, thermolysin, galactosidase, nucleases and trypsin.

However, none of above-mentioned methods alone is capable of producing a completely cell-free biological scaffold. Cell debris in the biological scaffold is known to induce severe host immune response. In addition, some decellularization treatments may damage the structure of ECM that further limits the application of thus produced biological scaffolds in tissue regeneration and/or remodeling.

In view of the foregoing, there exists in the related art a need for a novel method for efficiently removing cells from a tissue or an organ without destroying its structure, and accordingly, providing a safe and effective biological scaffold to treat a subject in need of tissue implantation.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, the first aspect of the disclosure is directed to a method of producing a decellularized bioscaffold from an organ or a vessel, in which the organ or the vessel is partially or fully covered by a lining tissue. The present method comprises,

(a) removing the lining tissue by applying a digestion buffer onto the outer surface of the organ or the vessel; and

(b) subjecting the product of the step (a) to a decellularization buffer comprising a decellularization agent so as to produce the decellularized bioscaffold.

According to some preferred embodiments of the present disclosure, in the step (a), the organ or the vessel is treated with the digestion buffer comprising a thickening agent and a digesting enzyme. Preferably, the digestion buffer is applied on the outer surface of the organ or the vessel. The purpose of adding thickening agent in the digestion buffer is to increase the viscosity (i.e., reduce the fluidity) of the digestion buffer, restricting the digestion to the outer surface of the organ or the vessel. That is, the lining tissue can thus be removed without damaging the underlying tissue/organ. According to the embodiments of the present disclosure, the digestion buffer has a viscosity suffice enough to prevent free-flow of the digestion buffer so that the digestion buffer remains on the outer surface of the organ or the vessel. According to one working example of the present disclosure, the viscosity of the digestion buffer is 8 centipoise (cP).

Depending on the nature of the organ or the vessel, the lining tissue may be the adventitia or the serosa. In general, blood vessels, lymphatic vessels and the retroperitoneal organs (e.g., thoracic esophagus, ascending colon, descending colon, the rectum, the gallbladder, the kidneys and the pancreas) are covered by the adventitia; while the intraperitoneal organs (e.g., the bladder, the liver, the heart, the uterus, the stomach and the intestine) are covered by the serosa.

The thickening agent of the digestion buffer is selected from the group consisting of, sucrose, dextran, starch, starch derivative, pectin, pectin derivative, alginic acid, alginate, gelatin, cellulose, cellulose derivative, galactomannan, xanthan, carrageen, karaya gum, tara gum, tamarind gum, gellan gum, mannan, maltodextrin, glycerol, poly(vinyl alcohol), polyurethane, and a combination thereof. According to some embodiments of the present disclosure, the thickening agent is sucrose.

According to certain embodiments of the present disclosure, the digesting enzyme is collagenase.

Then, in the step (b), the organ/tissue having the lining tissue (e.g., the adventitia or the serosa) removed is treated with the decellularization buffer selected from the group consisting of, sodium dodecyl sulfate (SDS), Triton X-100, Triton N-101, Triton X-114, Triton X-405, Triton X-705, Triton DF-16, monolaurate (Tween 20), monopalmitate (Tween 40), monooleate (Tween 80), ethylenediaminetetraacetic acid (EDTA), 3-[(3-cholamidopropyl)dimethylammonio]-1-propanesulfonate (CHAPS), 3-[(3-cholamidopropyl)dimethylammonio]-2-hydroxy-1-propansulfonate (CHAPSO), NP-40, sodium deoxycholate (SD), sodium cholate, N-lauroylsarcosine sodium salt, lauryldimethylamine-oxide (LDAO), cetyltrimethylammonium bromide (CTAB), cetylpyridinium chloride (CPC), octyl thioglucoside, octyl glucoside, dodecyl maltoside, perfluorononanoate, perfluorooctanoate, benzalkonium chloride (BAC), benzethonium chloride (BZT), nonoxynol-9, sulfobetaine-10 (SB-10), sulfobetaine-16 (SB-16), and a combination thereof. According to preferred embodiments of the present disclosure, the decellularization agent is SDS

The second aspect of the present disclosure pertains to a decellularized bioscaffold produced in accordance with the method of the first aspect and/or any of above embodiments. According to embodiments of the present disclosure, the present decellularized bioscaffold is capable of supporting the infiltration and growth of cells, for example, macrophages and fibroblasts.

Optionally, the present decellularized bioscaffold further comprises cells grown thereon. According to one embodiment, the cells are stem cells, progenitor cells, and/or differentiated cells.

The third aspect of the present disclosure is directed to a method of treating a subject in need of a bioscaffold implant. The present method comprises implanting the present decellularized bioscaffold into the subject. Depending on desired effects, the organ or the vessel for producing the present decellularized bioscaffold may be obtained from the subject himself/herself, an allogeneic subject or a xenogeneic subject.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIGS. 1A-1C are line charts that respectively depict the permeability (k) of the HUAs treated with 0.1% or 0.2% (w/v) collagenase solution containing 0% sucrose (FIG. 1A), 30% sucrose (FIG. 1B) and 50% sucrose (FIG. 1C) according to example 1.1 of the present disclosure. The HUAs with the intact adventitia were essentially water tight; the k value is zero. In the case of 120-min incubation in collagenase solution of low viscosity, the vessel wall of the HUA was damaged and hence no k values were reported.

FIG. 2 shows representative picro-sirius red (PSR) stained sections of the HUAs produced according to example 1.1 of the present disclosure. Panel a illustrates the HUAs prior to collagenase treatment. Panel b illustrates the HUAs treated with 0.2% (w/v) collagenase solution of high viscosity for 60 minutes, in which the adventitia of the HUAs was successfully removed by collagenase treatment, while the arterial wall remained intact. Panels c and d illustrate a PSR stained section of a human umbilical cord under polarized light and normal light, respectively. The scale bars respectively represent 100 μm in Panels a and b, and 2 mm in Panels c and d.

FIG. 3 shows representative H&E stained sections of the HUAs having the intact adventitia (Panels a, c, e and g) and the HUAs having the adventitia removed (Panels b, d, f and h) that were respectively decellularized by using 1% SDS with specified treatments for 48 hours according to example 1.2 of the present disclosure. The nuclei are marked by arrows. The scale bar represents 100 μm.

FIGS. 4A and 4B are diagrams respectively depicting the time courses of residual DNA in the HUAs having the intact adventitia and in the adventitia-free HUAs that were decellularized by using 1% SDS with specified treatments according to example 1.2 of the present disclosure. The data are presented as mean±standard deviation. N=3 per group per time point. * p<0.05; † p<0.001.

FIGS. 5A to 5D show the efficacy of different surfactants on removing cells in the adventitia-free HUAs produced according to example 1.3 of the present disclosure. FIG. 5A is a representative H&E stained section of the HUAs treated with 1% SDS for 24 hours. FIG. 5B is a representative H&E stained section of the HUAs treated with 4% SD for 72 hours. FIG. 5C is a representative H&E stained section of the HUAs treated with 3% Triton X-100 for 72 hours. FIG. 5D is the diagram depicting the levels of residual DNA in the HUAs respectively subjected to specified treatments. DI water: deionized water. The nuclei are marked by arrows. The scale bars respectively represent 100 μm.

FIGS. 6A to 6C are diagrams respectively depicting the mean pressure-diameter curves, the mean circumferential stress-stretch curves, and the compliance of the HUAs subjected to specified treatments according to example 1.4 of the present disclosure. dHUA: decellularized HUA. The data are presented as mean ±standard deviation. N=5 per group. *p<0.05.

FIGS. 7A and 7B are histograms respectively depicting the burst pressure and the suture retention strength of the HUAs subjected to specified treatments according to example 1.4 of the present disclosure. dHUA: decellularized HUA. The data are presented as mean ±standard deviation (S.D.). N=5 per group. * p<0.05.

FIGS. 8A and 8B are representative H&E stained and Masson's trichrome stained sections, respectively, of the decellularized HUAs (d-HUA) that were produced according to example 2 of the present disclosure and subcutaneously implanted into rats for specified periods of time. The scale bar represents 100 μm.

FIGS. 9A and 9B are representative H&E stained and Masson's trichrome stained sections, respectively, of the d-HUAs that were produced according to example 2 of the present disclosure and intraperitoneally implanted into rats for specified periods of time. The scale bar represents 100 μm.

FIG. 10 shows representative immunofluorescence of three macrophage markers in the sections of the d-HUAs that were produced according to example 2 of the present disclosure and subcutaneously implanted into rats for specified periods of time. The scale bar represents 200 μm.

FIG. 11 shows representative immunofluorescence of three macrophages markers in the sections of the d-HUAs that were produced according to example 2 of the present disclosure and intraperitoneally implanted into rats for specified periods of time. The scale bar represents 200 μm.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “lining tissue” refers to a layer of tissue covering an organ or a vessel externally. The term “lining tissue” encompasses the adventitia, the serosa, and any other structures that restrict water transport, in which the adventitia is a connective tissue covering the blood vessel, the lymphatic vessel, or the retroperitoneal organ (i.e., the organ in the abdominal cavity behind the peritoneum); while the serosa is a smooth tissue membrane covering the intraperitoneal organs (i.e., the organ in the abdominal cavity beyond the peritoneum). In general, the organs covered by the adventitia include, but are not limited to, thoracic esophagus, ascending colon, descending colon, the rectum, the gallbladder, the kidneys and the pancreas. Exemplary organs covered by the serosa include, but are not limited to, the bladder, the liver, the heart, the uterus, the stomach and the intestine.

The term “stem cell” as used herein refers to an undifferentiated cell, which is capable of self-maintenance or self-renewal (i.e., proliferation to give rise to more stem cells), and may give rise to lineage committed progenitors that are capable of differentiation and expansion into a specific lineage. The term “stem cell” refers to a generalized mother cell whose descendants (progeny) specialize, often in different directions, by differentiation, e.g., by acquiring completely individual characters, as occurs in progressive diversification of embryonic cells and tissues. In general, the stem cell can be obtained from embryonic, post-natal, juvenile, or adult tissue. The stem cell can be pluripotent or multipotent.

The term “progenitor cell” as used herein refers to an undifferentiated cell derived from a stem cell, and is not itself a stem cell. Some progenitor cells can produce progeny that are capable of differentiating into more than one cell type. A distinguishing feature of a progenitor cell is that, unlike a stem cell, it does not exhibit self-maintenance, and typically is thought to be committed to a particular path of differentiation and will, under appropriate conditions, eventually differentiate along this pathway.

As used herein, the term “differentiated cell” refers to a cell that has developed from an unspecialized phenotype to a specialized phenotype. Specifically, the term “differentiated cell” refers to a cell of a more specialized cell type (i.e., decreased developmental potential) derived from a cell of a less specialized cell type (i.e., increased developmental potential) (e.g., from a stem cell, a progenitor cell, an undifferentiated cell or a reprogrammed cell) where the cell has undergone a cellular differentiation process.

The term “surfactant” as used herein is given its ordinary meaning in the art and refers to compounds having an amphiphilic structure, which gives them a specific affinity for oil/water-type and water/oil-type interfaces, which helps the compounds to reduce the free energy of these interfaces and to stabilize the dispersed phase of a microemulsion. The term “surfactant” encompasses cationic surfactants, anionic surfactants, zwitterionic (amphoteric) surfactants, nonionic surfactants, and mixtures thereof. In general, nonionic surfactants do not contain any charges. Zwitterionic surfactants have both positive and negative charges; however, the net charge of the surfactant can be positive, negative, or neutral, depending on the pH of the solution. Anionic surfactants generally possess a net negative charge. Cationic surfactants generally possess a net positive charge.

As used herein, the term “thickening agent” has its usual meaning and is intended to denote substances, which, when added to an aqueous mixture, increase its viscosity without substantially modifying other properties.

As used herein, the term “viscosity” has its general meaning in the art. Specifically, the term “viscosity” as used herein may be the “kinematic viscosity” or the “absolute viscosity.” The “kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. If one fluid takes 100 seconds to complete its flow and another fluid takes 200 seconds, then the second fluid is twice as viscous as the first on a kinematic viscosity scale. Regarding the “absolute viscosity” (also known as “dynamic” or “simple viscosity”), it is the product of the kinematic viscosity and the fluid density. The dimension of the kinematic viscosity is L²/T where L is a length and T is a time. Commonly, the kinematic viscosity is expressed in centistokes (cSt). The international system of unit (SI unit) of the kinematic viscosity is mm²/s, which is 1 cSt. The absolute viscosity is expressed in units of centipoise (cP). The SI unit of absolute viscosity is the milliPascal-second (mPa-s), where 1 cP=1 mPa-s.

The viscosity of a solution (e.g., the present digestion buffer) can be increased by the addition of a thickening agent. The viscosity of a solution comprising a thickening agent is increased compared to that of a solution lacking the thickening agent. Depending on desired purposes, the thickening agent may increase the viscosity of a solution by about 5%, 10%, 15%, 20%, 25%, 30%, 35%, 40%, 45%, 50%, 55%, 60%, 65%, 70%, 75%, 80%, 85%, 90% or 95% compared to the viscosity of a solution without, ror containing lower amounts of the thickening agent.

The term “saturated” solution refers to a solution that contains the maximum amount of solute has been dissolved. Specifically, the term “saturated” solution as used herein refers to a solution containing a concentration of the digesting enzyme (e.g., collagenase) that is equal to the amount of the digesting enzyme (e.g., collagenase) that maximally can be dissolved at room temperature, the so-called “saturation concentration”.

As used herein, the term “outer surface” with respect to an organ or a vessel refers to the portion of the organ or the vessel that is exposed to air and visible. The term “outer surface” also includes a section of the organ or the vessel that resides just below the exposed, visible portion of the organ or the vessel. For example, when the present digestion buffer is applied to the outer surface of the organ or the vessel, some of the digestion buffer can be absorbed by the organ or the vessel so that some of the digestion buffer is below the exposed, visible surface of the organ or the vessel.

The term “implant” as used herein refers to the bioscaffold of the invention, which may be introduced into the body of a patient to replace or supplement the structure or function of the endogenous tissue.

As used herein, the term “autologous” refers to the cell, tissue or organ, which originates with or is derived from the recipient; and the term “allogeneic” refers to the cell, tissue or organ, which originates with or is derived from a donor of the same species as the recipient. The term “xenogeneic” refers to the cell, tissue or organ, which originates with or is derived from a species other than that of the recipient.

The term “subject” refers to a mammal including the human species that is treatable with methods of the present invention. The term “subject” is intended to refer to both the male and female gender unless one gender is specifically indicated.

The present disclosure aims at providing a decellularized bioscaffold with low immunogenicity and high efficacy in promoting tissue regeneration/remodeling. Accordingly, the first aspect of the present disclosure is directed to a method of producing a decellularized bioscaffold from a tissue or an organ covered by a lining tissue. The present method comprises the steps of,

(a) removing the lining tissue by applying a digestion buffer onto the outer surface of the organ or the vessel; and

(b) subjecting the product of the step (a) to a decellularization buffer comprising a decellularization agent so as to produce the decellularized bioscaffold.

In general, the lining tissue may be the adventitia or the serosa. Specifically, in the case when the organ is the bladder, the liver, the heart, the uterus, the stomach or the intestine, the lining tissue covered thereon is the serosa. The tissues or organs covered by the adventitia include, but are not limited to, blood vessels, lymphatic vessels, thoracic esophagus, ascending colon, descending colon, the rectum, the gallbladder, the kidneys and the pancreas. According to one working example of the present disclosure, the decellularized bioscaffold is produced from a blood vessel having the adventitia covered thereon.

In the step (a), the tissue or the organ is subject to a digestion buffer, which digests and removes the lining tissue therefrom. According to embodiments of the present disclosure, the digestion buffer comprises a thickening agent and a digesting enzyme, which, preferably, is a collagenase. The digesting enzyme is present in the digestion buffer for the purpose of digesting away the lining tissue, whereas the thickening agent is present for the purpose of keeping the digestion buffer at a preferred viscosity so as to restrict the digestion buffer from flowing freely. According to the preferred embodiment of the present disclosure, the digestion buffer has a viscosity sufficient enough to prevent free-flow of the digestion buffer so that the digestion buffer remains on the outer surface of the organ or the vessel.

Non-limiting examples of the thickening agents suitable for the present disclosure include, but are not limited to, sucrose, dextran, starch, starch derivative, pectin, pectin derivative, alginic acid, alginate, gelatin, cellulose, cellulose derivative, galactomannan, xanthan, carrageen, karaya gum, tara gum, tamarind gum, gellan gum, mannan, maltodextrin, glycerol, poly(vinyl alcohol), polyurethane, and a combination thereof. According to one working example, the thickening agent is sucrose.

Further, the thickening agent may be present at a concentration of 10-99% (w/v) in the digestion buffer, for example, 10, 11, 12,13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99%. According to some preferred example of the present disclosure, the thickening agent is sucrose, which is present at a concentration of at least 30% (w/v); more preferably, at a concentration of at least 50% (w/v). According to one working example of the present disclosure, the sucrose is present at a concentration of 50%. As would be appreciated, the viscosity of the digestion buffer depends on the concentration of the thickening agent. According to some embodiments of the present disclosure, the degree of the lining tissue being removed from the tissue or organ depends on the viscosity of the digestion buffer and the concentration of enzyme solution. According to some embodiments, the tissue or organ treated by a digestion buffer of low viscosity or a digestion buffer containing no thickening agent is highly permeable after such treatment, in which the ECM integrity of the tissue or organ is significantly damaged. According to other embodiments, the tissue or organ treated by a digestion buffer of high viscosity is relatively less permeable, yet the ECM integrity of the tissue or organ remain relatively intact, rendering the subsequent decellularization process effective to achieve a completely removal of residual cells.

Preferably, the viscosity of the digestion buffer is at least 1.0 cP; for example, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 15, 20, 25, 30, 35, 40, 45, 50, 55, 60, 65, 70, 75, 80, 85, 90, 95, 100, 200, 300, 400, 500, 600, 700 800, 900, 1,000 or more cP. More preferably, the viscosity of the digestion buffer is at least 3.0 cP. In one embodiment, the viscosity of the digestion buffer is about 3.0 cP. In one preferred embodiment, the viscosity of the digestion buffer is about 8.0 cP.

The concentration of the digesting enzyme in the digestion buffer may vary with the viscosity of the digestion buffer. Specifically, the concentration of the digesting enzyme in the digestion buffer may increase with the increase of the viscosity of the digestion buffer. As the viscosity of the present digestion buffer is sufficient to restrict the digestion buffer to the outer surface of the organ or the vessel, the concentration of the digesting enzyme can be from 0.1% (w/v) to a saturated solution (i.e., the maximal amount of the digesting enzyme that can be dissolved in the digestion buffer). According to one embodiment of the present disclosure, the viscosity of the digestion buffer is 1.0-8.0 cP, and the digesting enzyme is a collagenase; in this embodiment, the collagenase is present at a concentration of 0.01-10% (w/v) in the digestion buffer, such as 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10% (w/v); preferably, at the concentration of 0.05-1.0% (w/v); more preferably, at the concentration of 0.1-0.2% (w/v), such as 0.1, 0.11, 0.12, 0.13, 0.14, 0.15, 0.16, 0.17, 0.18, 0.19, and 0.2% (w/v). According to one embodiment, neither hyaluronidase nor elastinase is effective in removing the lining tissue from the tissue or the organ (e.g., HUAs).

According to preferred embodiments, the digestion buffer, preferably the digestion buffer of high viscosity, is applied on the lining tissue of the tissue or organ only so as to prevent underlying ECM structure from being digested. For example, in the case when the organ is an intestine, then such treatment shall not damage the mucosa, submucosa and muscular layer of the intestine. In the case when the tissue is a blood vessel, then such treatment shall not destroy the structure of the tunica media and tunica intima of the blood vessel.

As would be appreciated, the digestion buffer may be formulated as a gel so as to restrict the digesting enzyme to the outer surface of the organ or the vessel. Depending on desired purposes, the gel may comprise poly(alpha-hydroxy acids), poly(lacticle-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D-lactide, L-lactide, caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG (poly(d,l-lactide-co-glycolide), PLA-PLGA, poloxamer 407, PEG-PLGA-PEG triblock copolymers, SAIB (sucrose acetate isobutyrate) or the combination thereof. These one or more components allow the digesting enzyme to be released from the gel in a controlled and/or sustained manner. For example, the gel containing the digesting enzyme and a polymer matrix can be applied on the outer surface of the organ or the vessel, in which the polymer matrix breaks down over time (e.g., days, weeks or months) on the outer surface of the organ or the vessel thereby releasing the digesting enzyme. Thus the administration of the gel can be localized and occur over a period of time.

In the step (b), once the lining tissue is removed, the product of the step (a) (i.e., the adventitia-free or serosa-free tissue or organ) is subject to the treatment of a decellularization buffer so as to remove any cells/cellular contents residing in the tissue or organ. According to embodiments of the present disclosure, the decellularization buffer comprises a decellularization agent, which may be an acid, an alkaline, an anionic surfactant, a cationic surfactant, a zwitterionic (amphoteric) surfactant or a non-ionic surfactant. Preferably, the surfactant is selected from the group consisting of, sodium dodecyl sulfate (SDS), Triton X-100, Triton N-101, Triton X-114, Triton X-405, Triton X-705, Triton DF-16, monolaurate (Tween 20), monopalmitate (Tween 40), monooleate (Tween 80), ethylenediaminetetraacetic acid (EDTA), 3-[(3-cholamidopropyl)dimethylammonio]-1-propanesulfonate (CHAPS), 3-[(3-cholamidopropyl)dimethylammonio]-2-hydroxy-1-propansulfonate (CHAPSO), NP-40, sodium deoxycholate (SD), sodium cholate, N-lauroylsarcosine sodium salt, lauryldimethylamine-oxide (LDAO), cetyltrimethylammonium bromide (CTAB), cetylpyridinium chloride (CPC), octyl thioglucoside, octyl glucoside, dodecyl maltoside, perfluorononanoate, perfluorooctanoate, benzalkonium chloride (BAC), benzethonium chloride (BZT), nonoxynol-9, sulfobetaine-10 (SB-10), sulfobetaine-16 (SB-16), and a combination thereof. According to one specific example, the decellularization agent is SDS, which is present in the decellularization buffer at the concentration of 0.1-10% (w/v), such as 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, and 10% (w/v); preferably, the concentration is about 1% (w/v).

According to one embodiment of the present disclosure, the decellularization effect of SDS is greater than that of SD or Triton X-100.

Optionally, the decellularization buffer further comprises a protease inhibitor, which prevents the proteolysis of ECM proteins of the organ or tissue by matrix metalloproteinases (MMPs) and/or other proteases. The protease inhibitor may be selected from the group consisting of, serine protease, cysteine protease, aspartic protease, metalloprotease, thiol protease, exopeptidase and a combination thereof.

Further, neither the digestion buffer nor the decellularization buffer comprises an anti-oxidant, which is reported to cause DNA damage, death and mutagenicity of human cells.

According to certain embodiments of the present disclosure, in the step (b), the decellularization is conducted by immersing the entire tissue or organ having the lining tissue removed in the decellularization buffer followed by gently agitating the decellularization buffer on an orbital shaker at a speed not exceeding 100 rpm. According to some optional embodiments of the present disclosure, the decellularization treatment in the step (b) may be conducted by perfusing the decellularization buffer into the lumen of the lining tissue-free tissue or organ with or without transmural pressure. In one working example, the transmural pressure is 30 mmHg.

In general, the lining tissue (i.e., the adventitia or the serosa) forms a barrier for both the decellularization treatment and in vivo cell infiltration. According to some embodiments of the present disclosure, after the decellularization treatment, the cellular contents (e.g., the residual cells and cellular DNA) in the lining tissue-free tissue or organ is lower than that of the tissue or organ having the intact lining tissue. In addition, according to in vivo implantation embodiments, the number of cells (e.g., macrophages and/or fibroblasts) infiltrated into the tissue or organ having the lining tissue removed is higher than that of the organ or tissue having the intact lining tissue. Accordingly, compared with the bioscaffold produced by conventional treatments, the present decellularized bioscaffold having much lower levels of cellular contents therein provides a safer and more efficiently means to treat a subject in need thereof.

The second aspect of the present disclosure is directed to the decellularized bioscaffold produced by the present method. The present decellularized bioscaffold is capable of supporting the attachment, infiltration and growth of cells associated with tissue-regeneration and/or tissue remodeling (for example, stem cells, progenitor cells, and differentiated cells).

Optionally, the present decellularized bioscaffold further comprises cells grown thereon that further enhance the efficacy of the decellularized bioscaffold on tissue regeneration and/or tissue remodeling. The cells grown in the decellularized bioscaffold can be stem cells, progenitor cells, and/or differentiated cells. Exemplary stem cells include, but are not limited to, hematopoietic stem cells, embryonic stem cells, induced pluripotent stem cells, liver stem cells, neural stem cells, pancreatic stem cells, mesenchymal stem cells, and the combination thereof. In general, the progenitor cell is selected from the group consisting of, a hematopoietic progenitor cell, a neuronal progenitor cell, a mesenchymal stem cell, and the combination thereof. The differentiated cells can be any of fibroblasts, endothelial cells, immune cells (e.g., macrophages, dendritic cells, natural killer cells, natural killer T cells, and/or T cells), muscle cells, liver cells, brain cells, epithelial cells or the combination thereof. According to one specific example, the cells infiltrated in vivo in the decellularized bioscaffold are macrophages and fibroblasts.

According to one embodiment of the present disclosure, the present decellularized bioscaffold (produced from the tissue/organ having the adventitia/serosa removed therefrom) has lower stiffness, higher compliance, and lower suture retention strength as comparison to the bioscaffold produced from the tissue/organ with the intact adventitia/serosa covered thereon.

Another aspect of the present disclosure pertains to a method of treating a subject in need of a bioscaffold implant. The method comprises implanting the present decellularized bioscaffold into the subject.

Depending on desired effects, the tissue or organ used to produce the present decellularized bioscaffold may be isolated from the subject him/herself (i.e., an autologous source), from an allogeneic subject, or from a xenogeneic subject.

In general, the tissue or organ may be isolated from any suitable animal source, including human, other mammalian (e.g., mouse, rat, cat, dog, pig, cow, sheep, horse, monkey or chimpanzee), avian (e.g., chicken, turkey, duck or goose), reptile and amphibian.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

Materials and Methods

Preparation of Human Umbilical Arteries

Human umbilical cords were obtained from a local clinic with patients' consent. Immediately after childbirth, the cord was placed in cold Hank's Buffer Saline Solution (HBSS) and then transferred to the laboratory for processing. Briefly, intact HUAs were isolated from the Wharton's jelly using blunt dissection. The HUAs were divided into two parts; one part was subjected to decellularization or mechanical testing, and the other part was subjected to enzymatic treatment for specified periods of time thereby removing the adventitia prior to further processing or testing.

Removal of the Adventitia of Human Umbilical Arteries

To remove the adventitia, the HUAs were treated with a collagenase solution of low, moderate or high viscosity, which was prepared by mixing the respective ingredients listed in Table 1.

TABLE 1
Composition of the collagenase solution of low, moderate or high
viscosity

| | Collagenase | | HBSS | |
|---|---|---|---|---|
| | type I | Sucrose | (ml, final | Viscosity |
| Collagenase solution | (mg) | (g) | volume) | (centipoise, cP) |
| Low viscosity | 0.1%: 10 | 0 | 10 | ~1 |
| (0% sucrose) | 0.2%: 20 | | | |
| Moderate viscosity | 0.1%: 10 | 3 | 10 | ~3 |
| (30% sucrose) | 0.2%: 20 | | | |
| High viscosity | 0.1%: 10 | 5 | 10 | ~8 |
| (50% sucrose) | 0.2%: 20 | | | |

In the case when the HUAs were to be treated with collagenase solution of low viscosity, the HUAs were immersed and incubated therein at 37° C. in a humidified CO₂ incubator for 30, 60 or 120 minutes. In the case when the HUAs were to be treated with collagenase solution of moderate or high viscosity, the HUAs were cannulated with plugged luer adaptors using 5-O suture prior to spreading the collagenase solution of moderate or high viscosity evenly on the outer surface to ensure the enzyme digestion occurred only at the outer surface. The HUAs were incubated at 37° C. in a humidified CO₂ incubator for 30, 60 or 120 minutes.

Permeability of the Adventitia-Free Human Umbilical Arteries

The collagenase treated HUA was cannulated with luer adaptors at both ends. Upon expelling air in the lumen, one end of the HUA was plugged and the other end was connected to a bottle filled with deionized water. The HUA was then pressurized to 30 cm-H₂O by adjusting the water level in the bottle. Transmural flow was measured with transmural pressure fixed at 30 cm-H₂O. The permeability of the vessel wall of the collagenase treated HUA was calculated using Darcy's law, which described the fluid flow through porous media. The permeability (k) were calculated by the equation of,

$$k = \frac{QL μ}{A Δ P} ,$$

where A is the area of luminal surface of the HUA (m²), ΔP is the pressure drop across the vessel wall (mmHg), μ is the viscosity of the deionized water (Pa·s), Q is the transmural flow (m³/s), and L is the wall thickness measuring by a custom-made high frequency ultrasound (lateral/axial resolution=180/42 μm). The HUAs that were not treated with collagenase served as the control.

Decellularization of the Adventitia-Free Human Umbilical Arteries

Sodium dodecyl sulfate (SDS) was selected as the decellularization agent to evaluate the effect of the removal of the adventitia on the decellularization of HUAs. SDS (Mallinckrodt Baker, Phillipsburg, N.J.) was dissolved in deionized water to prepare 1% (w/v) SDS solution.

The adventitia-free HUAs were decellularized by the SDS solution under three different conditions: simple agitation, simple perfusion, and pressurized perfusion for specified periods of time. For the simple agitation group, the HUA was agitated with excessive volume of the SDS solution on an orbital shaker at the speed of 100 rpm at room temperature. For simple perfusion and pressurized perfusion groups, the HUA was cannulated into a custom-made decellularization system, with continuous circulation of 250 ml the SDS solution at room temperature. The decellularization system consists of a peristatic pump, a chamber that accommodates the HUAs to be decellularized, a feeding reservoir, a pressure transducer (Model 80A-005G, 0-5 psi, Sensormate, Taiwan) and a restrictor. The SDS solution was perfused at an average flow rate of 30 ml/min with a pulse frequency of 1 Hz. The transmural pressure will be set at 0 mmHg and 30 mmHg for the groups of simple perfusion and pressurized perfusion, respectively. After specified periods of time, the HUA was cut into 2-mm segments and washed in deionized water with agitation for at least five times until no bubbles were found in the solution to remove residual SDS, and then processed for either histology or DNA quantification. The HUAs having the intact adventitia were subjected to the same decellularization protocol as mentioned above, and the results were compared to that of the adventitia-free HUAs. Note that, to completely decellularize the HUAs having the intact adventitia, relatively short segments of the HUAs were used. The segment was immersed in the decellularization solution with simple agitation to ensure complete cell removal before the subsequent in vivo cell infiltration experiment.

Histology and DNA Quantification

The HUAs were fixed in 10% neutral-buffered formalin overnight at room temperature, dehydrated through a series of graded alcohol overnight, and then embedded in paraffin to enable examination of cross sections. Five micron sections were cut using a microtome (Leitz 1512, Leica, Germany) and collected on positively charged slides. After the removal of paraffin and re-hydration, sections were stained with H&E, Alcian blue, and picro-sirius red (PSR) for illustration of nuclei, glycosaminoglycans (GAGs), and collagen, respectively. Histological images were acquired by an optical microscope (DM2500P, Leica, Germany) with a CCD camera (DFC295 digital camera, Leica, Germany). In particular, PSR-stained sections were imaged under polarized light. Quant-iT PicoGreen dsDNA assay kit (Invitrogen, USA) was used to quantify residual DNA in the processed HUAs. Briefly, the specimen was lyophilized at −40° C. for 24 hours and its dry weight was measured. The dried specimen was incubated in a papain solution, which contains 20 U/mL papain (Worthinton, Lakewood, N.J.), 1.1 mM EDTA (Panreac, Spain), 5.5 mM cysteine-HCI (Panreac, Spain) and 0.067 mM 2-mercaptoethanol (Alfa Aesar, England) overnight at 60° C. until the specimen was completely digested. The solution was then diluted with 0.2 M Tris-EDTA buffer and then incubated with the working solution of the kit in a 96-well plate. The fluorescence of the sample was measured using a fluorometer (excitation: 485 nm, emission: 538 nm; Fluoroskan Ascent, Thermo Fisher Scientific, Waltham, Mass.) and values were compared with a λ dsDNA standard (0˜10 ng/mL) to determine the weight of the residual DNA. Finally, the weight of the residual DNA was normalized by the dry weight.

Upon the evaluation of the effect of the removal of the adventitia on the decellularization of HUAs using the SDS solution, two other surfactants, SD and Triton X-100, were tested for their efficacy acting as a decellularization agent to HUAs. SD and Triton X-100 were respectively dissolved in deionized water thereby preparing 4% (w/v) SD solution and 3% (w/v) Triton X-100 solution. The adventitia-free HUAs were decellularized respectively by the SD solution and the Triton X-100 solution with pressurized perfusion for specified periods of time. The adventitia-free HUAs treated with deionized water (DI water) served as the control group. The efficacy of the decellularization was examined by histology and DNA quantification.

Mechanical Properties of the HUAs

The mechanical properties of the HUAs having the intact adventitia, the adventitia-free HUAs, and the adventitia-dree HUAs subjected to different decellularization conditions were examined by pressure-diameter tests using a custom-built mechanical tester, which consists of a stepper motor with a motion control system (MID-7604 and PXI-7330, National Instruments, Austin, Tex.), a syringe pump (KDS-210, KD Scientific, Holliston, Mass.), a pressure transducer (Model 209, 0-5psig, Setra, Boxborough, Mass.), a load cell (LTS-200GA, Kyowa, Japan), a 1394 CCD camera (656×492, Stingray F033B, Germany) with a TV lens (HF25HA-1B, Fujinon, Japan), and a custom-made loading frame. Pressure-diameter tests have been regarded as the best method to assess the mechanical properties of blood vessels as the tubular structure of the vessel is preserved. HUAs from five donors were used for mechanical testing. A sufficiently long HUA from each donor was cut into four segments of ˜25 mm long; each of which underwent one of the four treatments prior to mechanical testing. Briefly, the HUA was cannulated with luer adaptors using 5-O suture and coupled to the loading frame. Note that, the adventitia-free HUAs were tested with a PDMS tube inserted in the lumen of the HUA since water leakage from the vessel was expected during mechanical testing. The HUA was submerged in a chamber filled with normal saline at room temperature and then air in the tubing was expelled. The HUA was pressurized cyclically between 0 and 150 mmHg for ten times using a syringe pump at a flow rate of 0.2 ml/min for preconditioning. After preconditioning, the HUA was decoupled from the loading frame and recoupled at its unloaded configuration (the luminal pressure was about 10 mmHg, and the axial load was about 0 mN). The dimensions of the HUA (i.e., the outer radius and the length between the two suture ties) were recorded at the unloaded configuration; circumferential and axial stretches were calculated based on these dimensions. The axially constrained HUA was then subjected to cyclic pressurization at its unloaded length. Data from the loading phase of the cycle were analyzed for the stiffness of the HUA.

The compliance of the HUA was calculated by equation (i),

$$Compliance ( \% per 100 mmHG ) = \frac{( {D}_{1} - {D}_{2} )}{{D}_{2} ( {P}_{1} - {P}_{2} )} \times {10}^{4} , \qquad ( i )$$

where Pᵢ and Dᵢ (i=1 or 2) respectively represented the transmural pressure and the corresponding outer diameter of the HUA. Herein, the compliance was calculated between 70 and 120 mmHg. That is, P₁=120 mmHg and P₂=70 mmHg. As the compliance may be influenced by the wall thickness of the HUA, it is more informative to examine mechanical behavior of the HUA in terms of stress-stretch relationship.

The stress was determined based on the dimensions of the HUA at its deformed state. The unloaded thickness of the HUA, H, was measured from the histological section of the HUA using Image J (NIH). The wall volume (V) of the HUA was then determined by equation (ii),

V=π(B²−A²)L (ii),

where B represented the unloaded outer radius, A(=B−H) represented the unloaded inner radius, L represented the unloaded length, and H represented the unloaded thickness of the HUA.

Although not measurable, the deformed inner radius, a, at any deformed state can be computed by equation (iii) given the on-line measurement of deformed outer radius, b, with the assumption of incompressibility.

$$a = \sqrt{{b}^{2} - ( \frac{{B}^{2} - {A}^{2}}{{λ}_{z}} )} , \qquad ( iii )$$

where λ_{z} represented the axial stretch ratio

$$( \frac{l}{L} )$$

and l represented the deformed length of the HUA.

Once a and b were known, the mean circumferential stress, σ_{θ}, was then calculated in accordance with the pressure-diameter data by equation (iv),

$${σ}_{θ} = \frac{Pa}{h} , \qquad ( iv )$$

where P represented the transmural pressure, and h(=b−a) represented the deformed wall thickness.

The associated mean circumferential stretch ratio, μ₀, was determined by equation (v),

$${λ}_{θ} = \frac{( a + b ) / 2}{( A + B ) / 2} , \qquad ( v )$$

where a represented the deformed inner radius, b represented the deformed outer radius, A represented the unloaded inner radius, and B represented the unloaded outer radius.

The burst pressure and the suture retention strength were examined using the same custom-built mechanical tester except that a 100 psi pressure transducer (Model 80A, 0-100 psi, Sensormate, Taiwan) and a 50 N load cell (MOB-10, Transducer Technique, Temecula, Calif.) were used as larger maximum pressure and force were expected. The burst pressure of the HUAs, the maximum load that the specimen could bear before failure, was measured after preconditioning. Suture retention tests were performed on ˜20-mm long HUA segments by placing a 6-0 polypropylene suture approximately 2 mm from one end of the segment. With the suture connected to the load cell, the other end of the segment was fixed to the loading frame by cannulation. As the segment was stretched at an extension rate of 0.25 mm/s, the force was recorded until the suture pulled through the segment.

In Vivo Cell Infiltration

Fifteen male Sprague-Dawley rats (10 weeks-old) were used in this example. The rats were anesthetized intraperitoneally with 0.5 mg/kg Zoletil 50 (Virbac, Carros, France). All animals received humane care in compliance with the principles of laboratory animal care formulated by the Ethical Committee for Animal Research of the Show Chwan Memorial Hospital. Prior to implantation, a silicon tube was inserted into the lumen of the decellularized HUA to sustain the tubular shape of the HUA, and to ensure that cell infiltration starts from the outer surface of the vessel. The HUAs were implanted into both abdominal cavity and subcutaneous space in each rat. The implanted HUAs were retrieved 7 days (N=5), 14 days (N=5), and 28 days (N=5) after implantation.

Immunohistochemistry

The retrieved HUAs were fixed, processed, and embedded in paraffin. Five micron sections were prepared for histological examination. Specifically, after removal of paraffin and re-hydration, sections were immunostained for pan-macrophage (mouse anti-CD68), M1 macrophage (rabbit anti-CCR7), or M2 macrophage (goat anti-CD206). The sections were subject to microwave radiation in 1 mM ethylenediaminetetraacetic acid (EDTA) buffer, pH=8, for antigen retrieval followed by incubation with primary antibody (dilution rate: 1:25 for CD68, abcam, UK, 1:100 for CCR7, abcam, UK, and 1:25 for CD206, Santa Cruz, Dallas, Tex., 1:5000 for CD31, abcam, UK) for 1 hour at room temperature. After 3 times of PBS wash, the sections were incubated with a fluorescently labeled secondary antibody; AlexaFluor donkey anti-mouse IgG (350 nm) at a dilution of 1:25, donkey anti-rabbit IgG (568 nm) at a dilution of 1:100, and donkey anti-goat IgG (488 nm) at a dilution of 1:100 (Thermo Fisher Scientific, Waltham, Mass.), for 1 hour at room temperature, followed by DAPI counter staining to the nuclei for 30 minutes. Fluorescence images were acquired by a fluorescence microscope (Olympus BX 60, Olympus, Japan).

Statistical Analysis

Data was presented as mean ±standard deviation. Differences in mechanical properties of the HUAs having the intact adventitia, the adventitia-free HUAs, and the decellularized HUAs were examined by two-way ANOVA with repeated measures in conjunction with Tukey post-hoc procedure. Significance level was set at p<0.05.

### Example 1 Preparation and Characterization of Decellularized HUAs

1.1 Preparation of Adventitia-Free HUAs

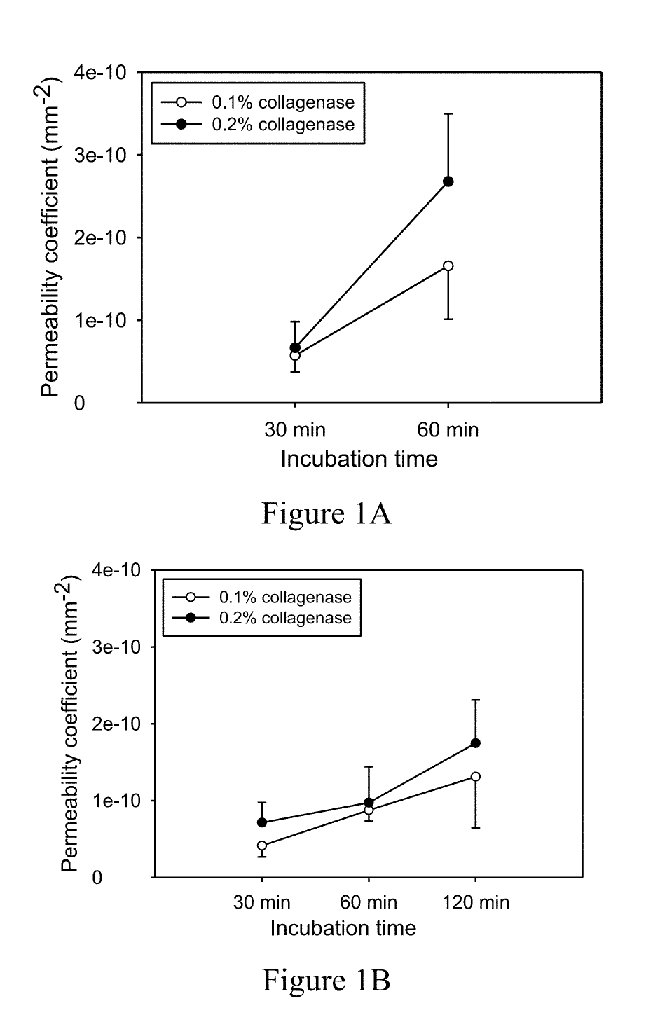

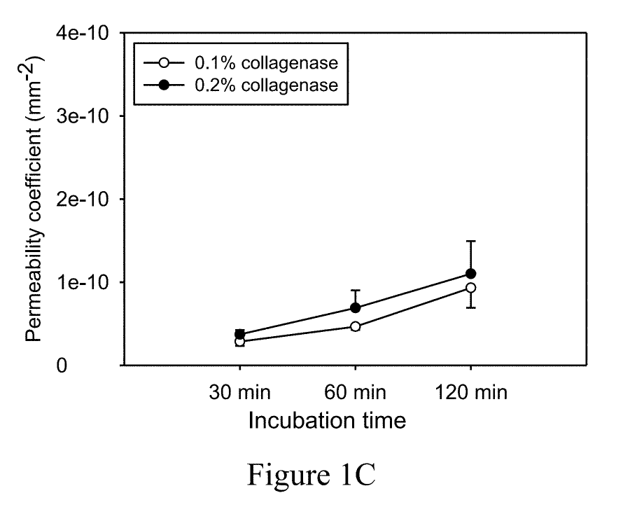

To remove the lining tissue of HUAs, the HUAs were subjected to the treatment of collagenase solution of low, moderate or high viscosity for 30, 60 or 120 minutes as descried in “Materials and Methods” section, and the effect of the treatment was evaluated by measuring the permeability of the collagenase-treated HUAs. Results were illustrated in FIGS. 1A-1C.

It was found that immersing the HUAs in 0.1% or 0.2% (w/v) collagenase solution of low viscosity significantly damaged the structure of the vessel wall, rendering the vessel highly water permeable (FIG. 1A). For the HUAs treated with 0.1% or 0.2% (w/v) collagenase solution of moderate viscosity, the value of permeability increased with an increase in the collagenase concentration or treatment time (FIG. 1B). Nevertheless, treating the HUAs with 0.2% (w/v) collagenase solution of high viscosity for 60 minutes more consistently produced a vessel having substantial permeability without losing its structure integrity (FIG. 1C).

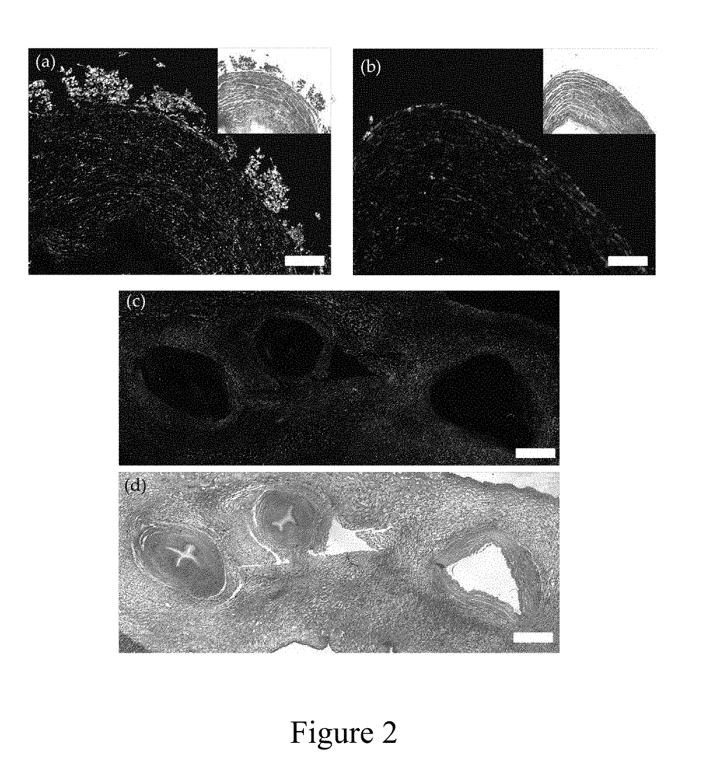

PSR staining further confirmed that, as compared with un-treated HUAs (FIG. 2, Panels a, c, and d), the HUAs treated with 0.2% (w/v) collagenase solution of high viscosity for 60 minutes were completely free of the adventitia (FIG. 2, Panel b), and the arterial wall remained relatively intact after collagenase treatment, indicating that the digestion was restricted to the outer surface of the vessel, probably due to the reduced fluidity of the collagenase solution.

Further, it was worth to note that neither hyaluronidase nor elastinase could produce adventitia-free HUAs as those treated with highly viscous collagenase solution (data not shown).

1.2 Effects of Permeability on Decellularization

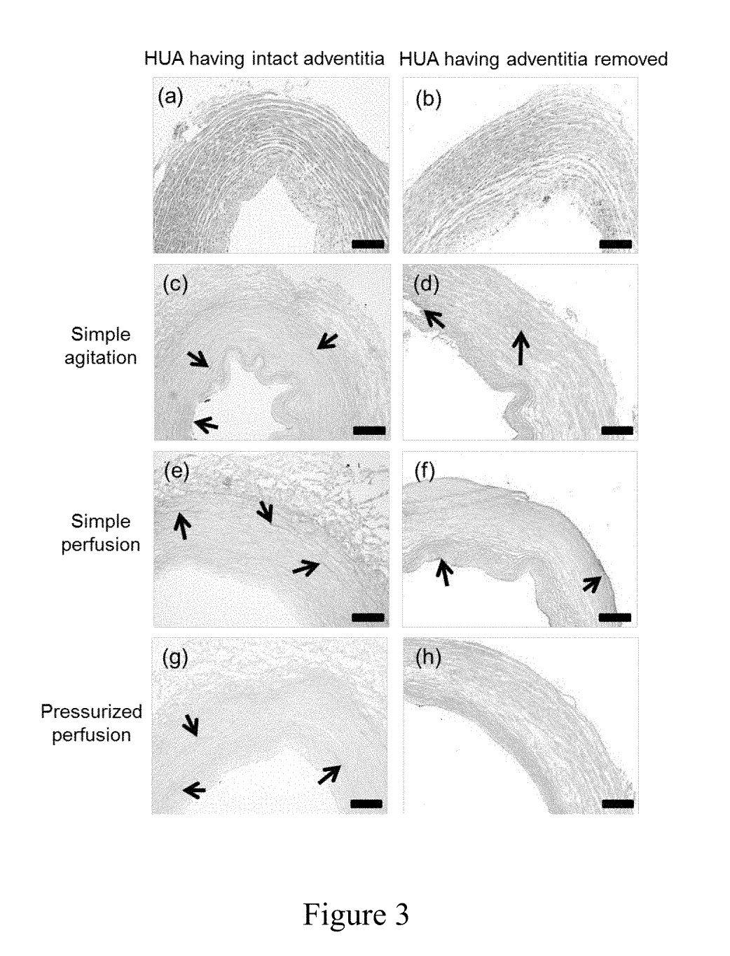

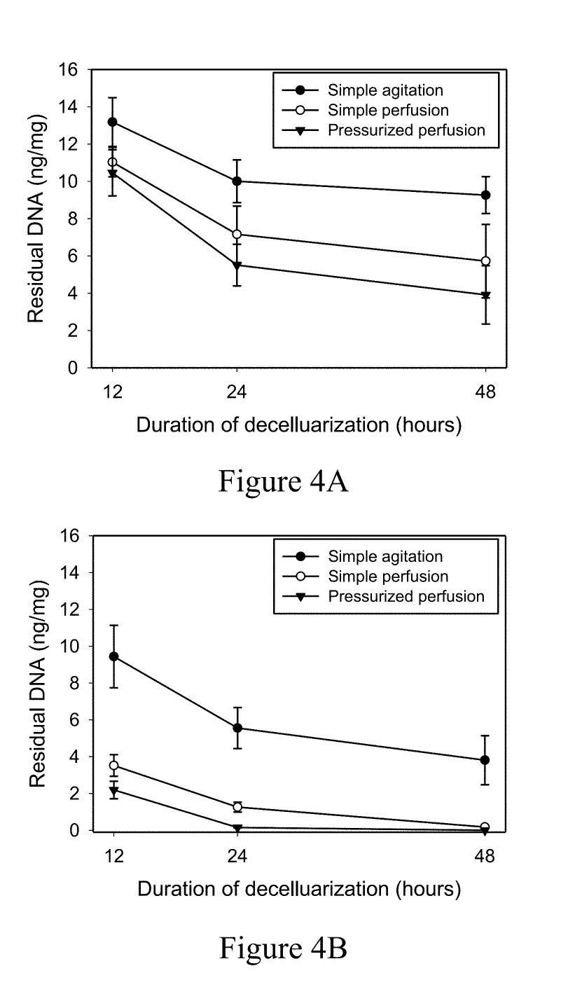

The effect of the removal of the adventitia of the HUAs on decellularization were examined by histology (H&E staining) and DNA quantification. The results were depicted in FIGS. 3 and 4.

Similar to the PSR results depicted in FIG. 2, H&E staining also confirmed that the HUAs treated with collagenase solution of high viscosity (0.2%, w/v, 60 minutes) were free of the adventitia, and the media remained relatively intact (FIG. 3, Panel b). After being treated with SDS solution, the number of blue spots of residual nucleus in the adventitia-free HUAs (indicated by arrows, FIG. 3, Panels d, f and h) was obviously lower than that of the HUAs having the intact adventitia (indicated by arrows, FIG. 3, Panels c, e and g).

The finding was further confirmed by DNA quantification. Given the same decellularization conditions, the level of residual DNA in the adventitia-free HUAs was significantly lower than that in the HUAs having the intact adventitia (FIGS. 4A and 4B). Furthermore, the level of residual DNA in the HUAs decellularized by pressurized perfusion was lower than that in the HUAs decellularized by simple perfusion or by simple agitation (FIGS. 4A and 4B). Particularly, for the adventitia-free HUAs, no residual DNA was found in the group of pressurized perfusion after 24 hours of decellularization (FIG. 4B).

Taken together, the data indicated that the removal of the adventitia improved the efficiency of decellularization.

1.3 Preparation of Decellularized HUAs

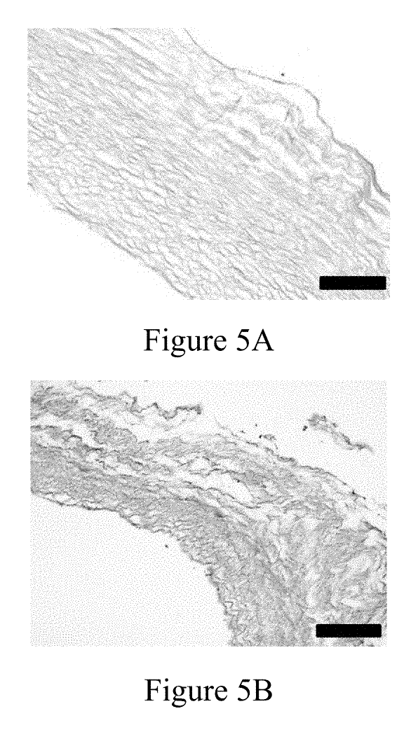

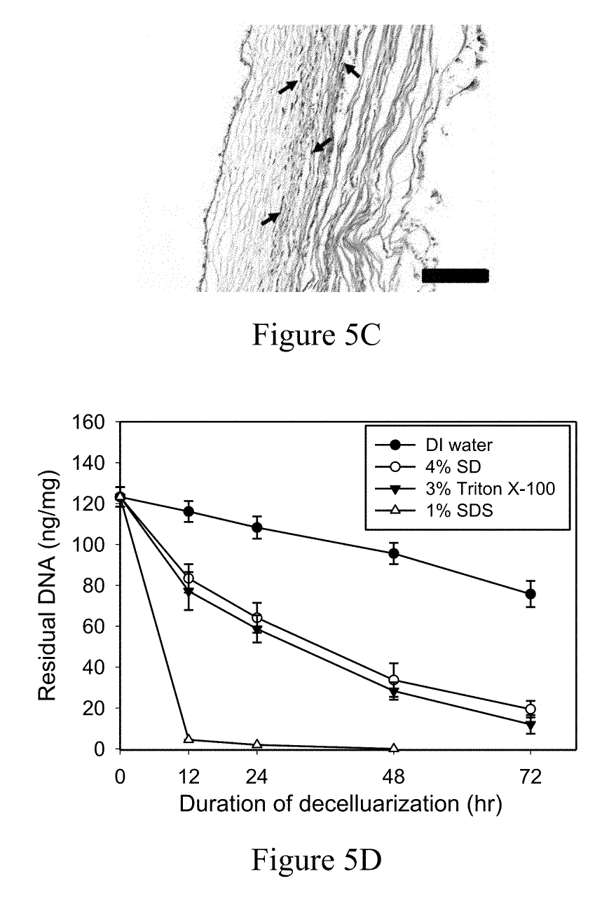

To examine the efficacy of other surfactants on decellularization of the adventitia-free HUAs of example 1.1, the HUAs were respectively treated with 1% SDS for 24 hours (FIG. 5A), 4% SD for 72 hours (FIG. 5B), and 3% Triton X-100 for 72 hours (FIG. 5C). As shown in FIG. 5D, the level of residual DNA in the HUAs decellularized by 1% SDS was significantly lower than that in the HUAs decellularized by 4% SD, 3% Triton X-100, or deionized water (DI water).

After determining the efficacy of specified surfactants on decellularization, the adventitia-free HUAs were treated with 1% SDS solution for 24 hours to remove cells in the tunica media for future applications in accordance with the procedures described in the “Materials and Methods” section.

1.4 Mechanical Properties of the Adventitia-Free HUAs of Example 1.1 and the Decellularized HUAs of Example 1.3

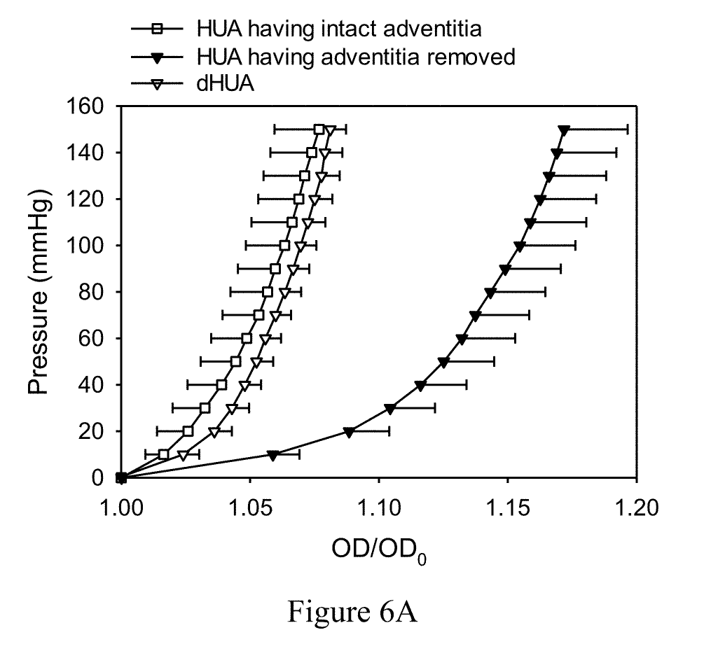

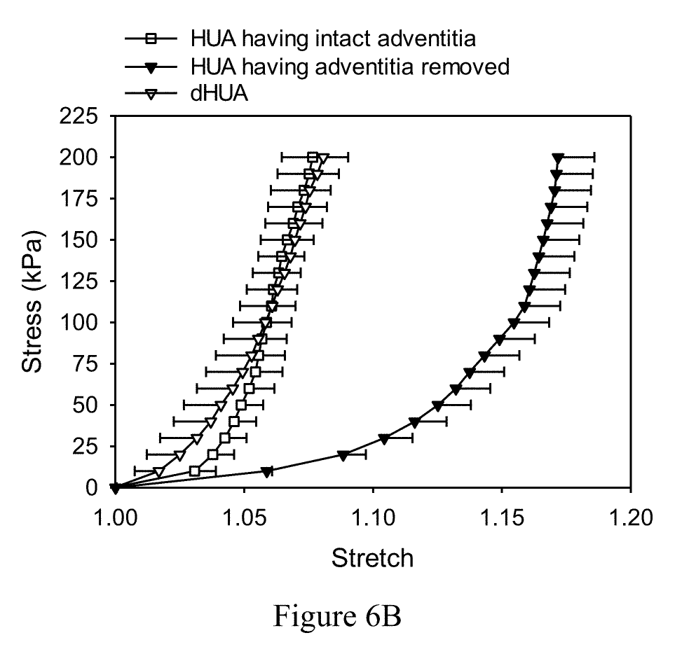

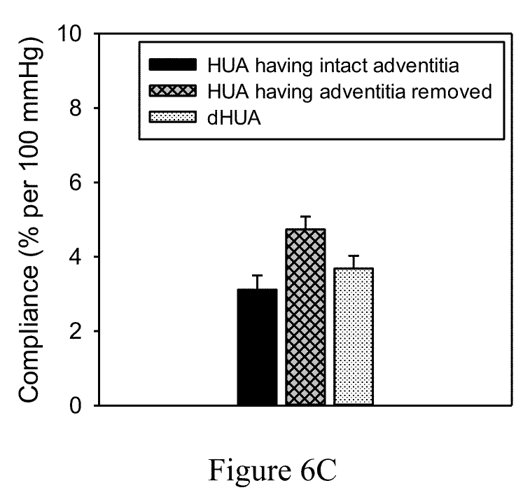

The mechanical properties of the decellularized HUAs of example 1.3, such as stiffness, compliance and suture retention strength, were analyzed in this example. FIG. 6A illustrated the pressure-diameter curves of the HUAs before and after the removal of the adventitia (the HUAs having the intact adventitia vs. HUAs having the adventitia removed; N=5). The mean pressure-diameter curve of the HUAs significantly shifted to the right after the removal of the adventitia; in other words, the HUAs having the intact adventitia manifested a stiffer mechanical behavior than the adventitia-free HUAs. Note that the pressure-diameter curve of a blood vessel represents its structural stiffness, which is influenced by the wall thickness of the vessel. Stress-stretch plots were prepared for the comparison of material stiffness, which is independent of the wall thickness. FIG. 6B illustrated the mean stress-stretch curves of the HUAs before and after the removal of the adventitia, in which significant differences were found between the mean stress-stretch curves. The data also indicated that the material stiffness decreased significantly after the removal of the adventitia. FIGS. 6A and 6B further illustrated the structural stiffness and the material stiffness of the adventitia-free HUAs before and after treated with different decellularization protocols (N=5 in each groups). Both mean pressure-diameter and mean stress-stretch curves shifted to the left after decellularization, which suggested that decellularization process increased the stiffness of the HUAs. The compliance of the HUAs having the adventitia removed was higher than that of the HUAs having the intact adventitia and dHUA (i.e., HUAs having the intact adventitia and decellularized HUA) (FIG. 6C).

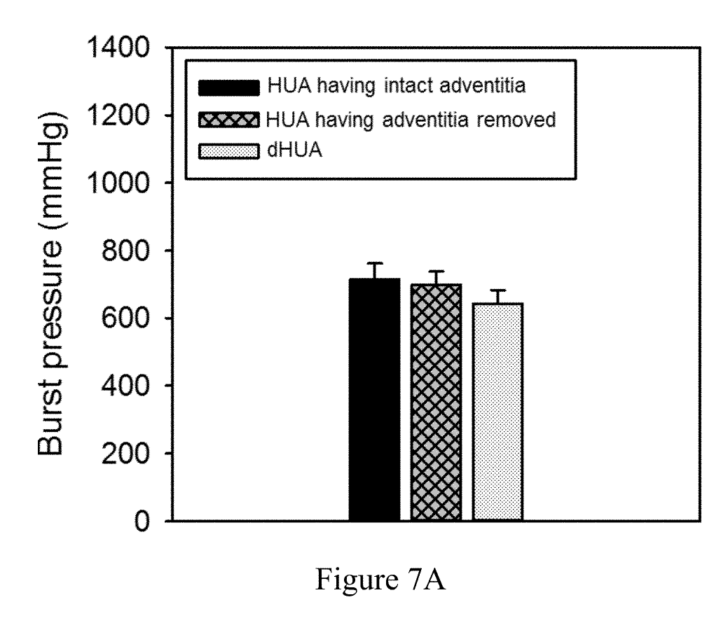

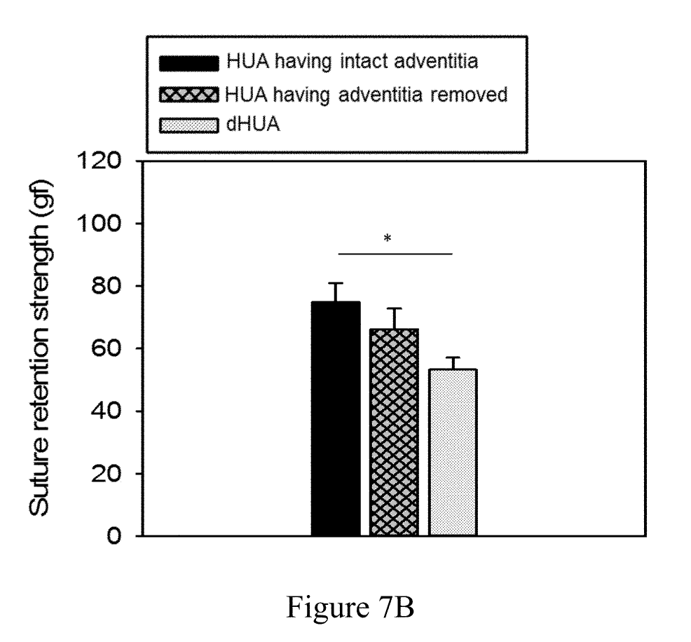

FIGS. 7A and 7B respectively illustrate the burst pressure and the suture retention strength of the HUAs having the intact adventitia, the HUAs having the adventitia removed, and the decellularized HUAs (dHUA), in which SDS solution was introduced by pressurized perfusion. No significant differences were found among the tested groups in burst pressure (FIG. 7A). However, compared to the HUAs having the intact adventitia, the suture retention strength decreased in HUAs having the adventitia removed (FIG. 7B).

These data indicated that the adventitia-free HUAs exhibited lower stiffness, higher compliance, and lower suture retention strength, as compared with the HUAs having the intact adventitia. In addition, compared with the adventitia-free HUAs prior to the decellularization process (i.e., the respective “HUA having the adventitia removed” groups in FIGS. 6 and 7), decellularization process increased the stiffness of the HUAs, yet decreased the compliance and the suture retention strength of the HUAs.

### Example 2 In Vivo Cell Infiltration

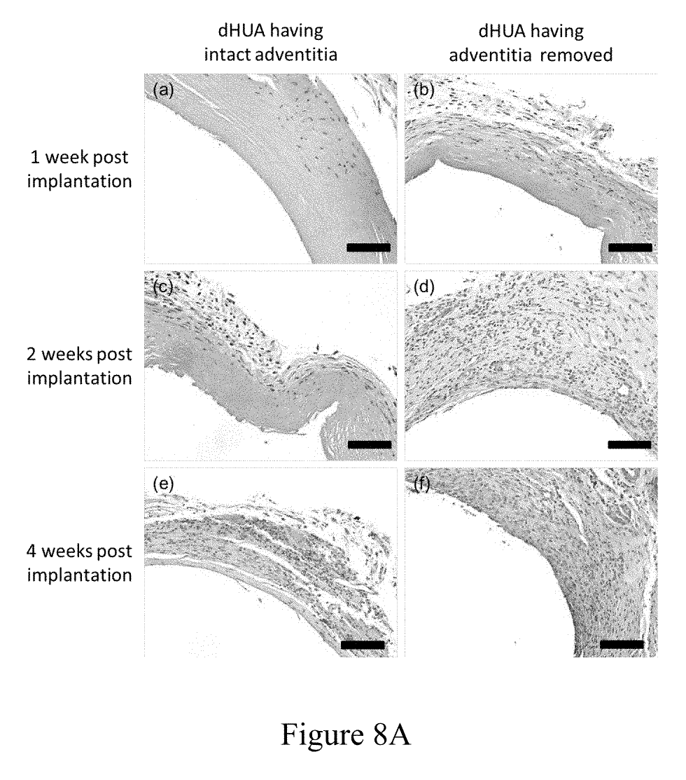

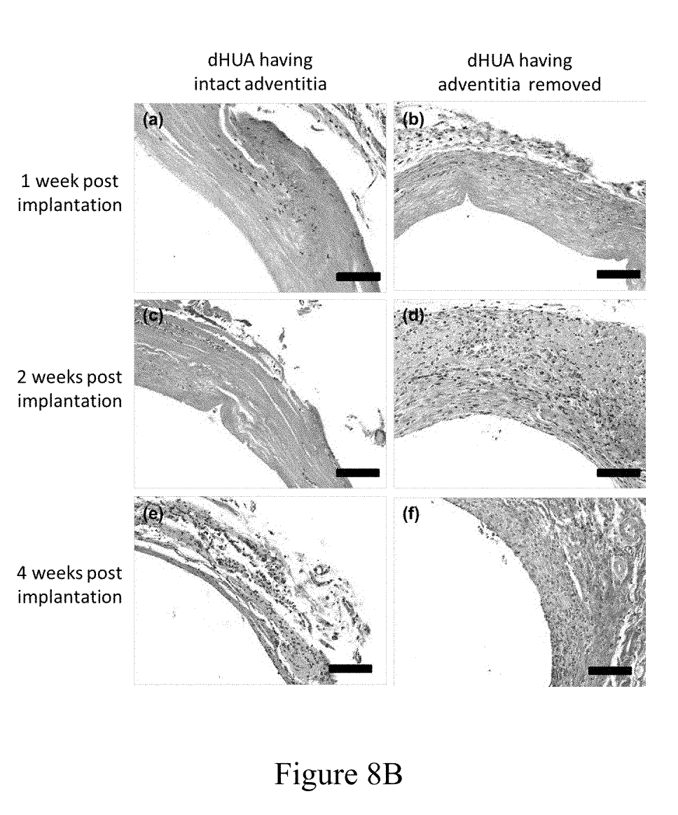

To investigate the efficacy of the decellularized HUAs of example 1 as a bioscaffold, the HUAs were implanted subcutaneously or intraperitoneally in animals as described in the “Materials and Methods” section. Short segments of the HUAs with the intact adventitia that were decellularized by immersing in 1% SDS solution with simple agitation served as the control. Results were illustrated in FIGS. 8 to 11.

FIGS. 8A and 8B are photographs of the histological staining of the decellularized HUAs taken after being implanted for 1 week (panels a and b), 2 week (panels c and d), or 4 weeks (panels e and f) in the subcutaneous space. Cells were found to stay in the outer area of the vessel in the sections of the decellularized HUAs having the intact adventitia, and tissue degradation was found at 4 weeks post-implantation. For the sections of the decellularized HUAs having the adventitia removed, cells were found in the exterior of the vessel at first week and then infiltrated into the tunica media after 2 weeks of implantation, in which neo-collagen was found in the sections at 2 weeks and 4 weeks post-implantation.

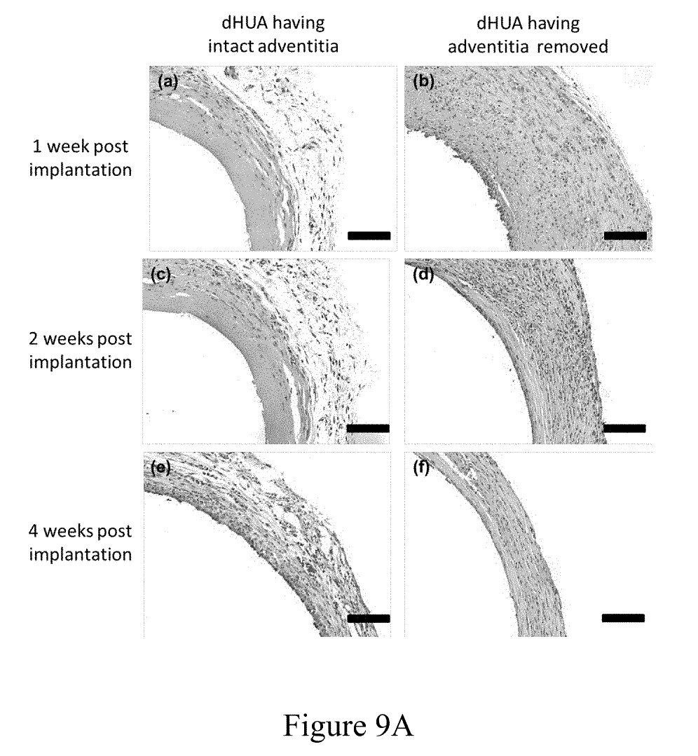

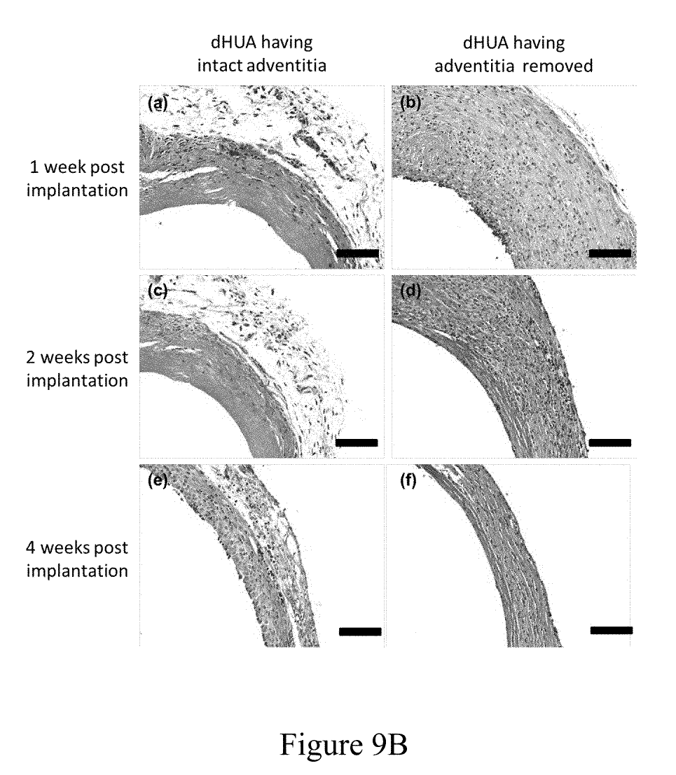

Similar findings were found in the implants of the abdominal cavity. Compared to the HUAs having the intact adventitia, in which cells were kept in the exterior of the vessel in the sections and did not infiltrate into the tunica media until 4th week post-implantation, cells were infiltrated into the tunica media of the HUAs having the adventitia removed at first week post-operation (FIGS. 9A and 9B, panels a and b). The depth and density of infiltrated cells were relatively greater in the decellularized HUAs having the adventitia removed. Newly formed collagen was found in the sections of the decellularized HUAs having the adventitia removed after 2 weeks and 4 weeks of implantation ((FIGS. 9A and 9B, panels c to f), while tissue degradation was observed in the decellularized HUAs having the intact adventitia.

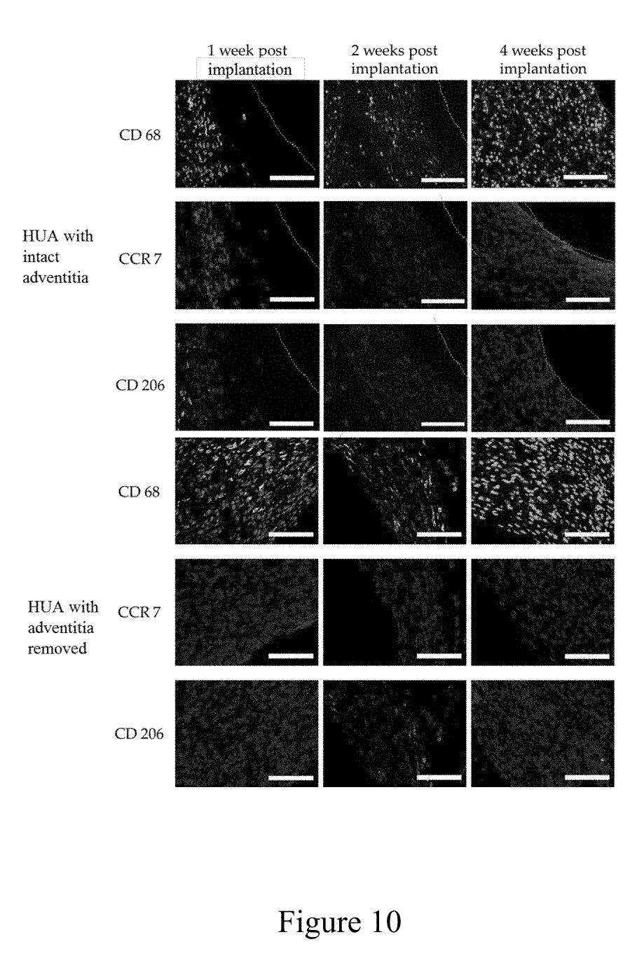

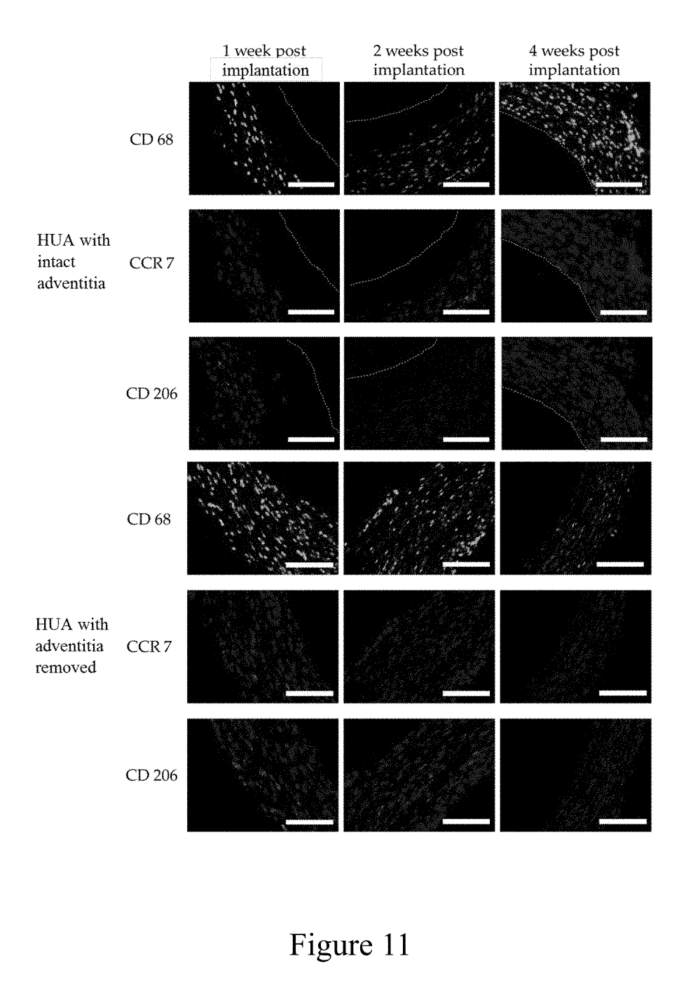

The presence and localization of macrophages in the sections of the implanted HUAs were confirmed by immunofluorescence (FIGS. 10 and 11). The results were consistent with the histological data described above. The macrophages remained at the outer area of the vessel in the sections of the decellularized HUAs having the intact adventitia; conversely, macrophages infiltrated into the tunica media in the sections of the decellularized HUAs having the adventitia removed. M1 macrophages dominated the immune response in the HUAs having the intact adventitia throughout the 4 weeks of implantation, especially in the subcutaneous implantation (FIG. 11). Contrary to the results of the HUAs having the intact adventitia, M2 macrophages dominated the immune response after 2 weeks of implantation in the HUAs having the adventitia removed and few macrophages were found at 4 week post-operation (FIGS. 10 and 11).

In conclusion, the present disclosure provides a novel method for producing a decellularized tissue (e.g., a vessel) suitable for use as a bioscaffold. Compared with conventional methods, the present method characterizes in that an enzyme solution (e.g., collagenase solution) of high viscosity is first used to remove the lining tissue (e.g., the adventitia of HUAs) from the biomaterial (e.g., the HUA), without damaging the interior structure of the biomaterial (e.g., the tunica media and tunica intima of the HUA). The removal of the adventitia/serosa significantly improves the efficacy of the subsequent decellularization treatment. The thus-produced decellularized bioscaffold contains low levels of cellular content, and is capable of efficiently supporting the infiltration, adhesion and growth of macrophages and fibroblasts, all of which participate in the tissue remodeling/tissue regeneration.

It will be understood that the above description of embodiments is given by way of example only and that various modifications may be made by those with ordinary skill in the art. The above specification, examples and data provide a complete description of the structure and the use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of producing a decellularized bioscaffold from an organ or a vessel, in which the organ or the vessel is covered by a lining tissue, the method comprises
(a) removing the lining tissue by applying a digestion buffer onto the outer surface of the organ or the vessel; and
(b) subjecting the product of the step (a) to a decellularization buffer comprising a decellularization agent so as to produce the decellularized bioscaffold;
wherein the digestion buffer comprises a thickening agent and a digesting enzyme, and has a viscosity sufficient enough to prevent free-flow of the digestion buffer so that the digestion buffer remains on the outer surface of the organ or the vessel.

2. The method of claim 1, wherein the lining tissue is adventitia.

3. The method of claim 1, wherein the thickening agent is selected from the group consisting of, sucrose, dextran, starch, starch derivative, pectin, pectin derivative, alginic acid, alginate, gelatin, cellulose, cellulose derivative, galactomannan, xanthan, carrageen, karaya gum, tara gum, tamarind gum, gellan gum, mannan, maltodextrin, glycerol, poly(vinyl alcohol), polyurethane, and a combination thereof.

4. The method of claim 3, wherein the thickening agent is sucrose.

5. The method of claim 1, wherein the digesting enzyme is collagenase.

6. The method of claim 1, wherein the decellularization agent is selected from the from consisting of, sodium dodecyl sulfate (SDS), Triton X-100, Triton N-101, Triton X-114, Triton X-405, Triton X-705, Triton DF-16, monolaurate (Tween 20), monopalmitate (Tween 40), monooleate (Tween 80), ethylenediaminetetraacetic acid (EDTA), 3-[(3-cholamidopropyl)dimethylammonio]-1-propanesulfonate (CHAPS), 34(3-cholamidopropyl)dimethylammonio]-2-hydroxy-1-propansulfonate (CHAPSO), NP-40, sodium deoxycholate (SD), sodium cholate, N-lauroylsarcosine sodium salt, lauryldimethylamine-oxide (LDAO), cetyltrimethylammonium bromide (CTAB), cetylpyridinium chloride (CPC), octyl thioglucoside, octyl glucoside, dodecyl maltoside, perfluorononanoate, perfluorooctanoate, benzalkonium chloride (BAC), benzethonium chloride (BZT), nonoxynol-9, sulfobetaine-10 (SB-10), sulfobetaine-16 (SB-16), and a combination thereof.

7. The method of claim 6, wherein the decellularization agent is SDS.

8. The method of claim 1, wherein the organ is thoracic esophagus, ascending colon, descending colon, rectum, gallbladder, kidney, pancreas, bladder, liver, heart, uterus, stomach or intestine.

9. A decellularized bioscaffold produced by the method of claim 1.

10. The decellularized bioscaffold of claim 9, further comprising cells grown thereon, wherein the cells are progenitor cells, stem cells, differentiated cells, and a combination thereof.

11. A method of treating a subject in need of a bioscaffold implant, comprising implanting the decellularized bioscaffold of claim 10 into the subject.

12. The method of claim 11, wherein the organ or the vessel is obtained from the subject himself/herself.

13. The method of claim 11, wherein the organ or the vessel is obtained from an allogeneic subject.

14. The method of claim 11, wherein the organ or the vessel is obtained from a xenogeneic subject.
